(19) United States
(12) Patent Application Publication
(10) Pub. No.: US 2020/0354659 A1
(43) Pub. Date: Nov. 12, 2020

(54) Photoreactor for Cell Assay and Array Chemical and Biochemical Reactions

(71) Applicant: Michael Jared Willis, Richmond, TX (US)

(72) Inventor: Michael Jared Willis, Richmond, TX (US)

(21) Appl. No.: 16/765,809

(22) PCT Filed: Nov. 20, 2018
(86) PCT No.: PCT/IB2018/059149

§ 371 Date: May 20, 2020

Related U.S. Application Data
(60) Provisional application No. 62/588,788, filed on Nov. 20, 2017.

(51) Int. Cl. C12M 1/00; C12M 1/02; C12M 1/36

(52) U.S. Cl. CPC C12M 21/02; C12M 31/08; C12M 41/48; C12M 41/18; C12M 31/06

(57) ABSTRACT

A photoreactor for cell assay and array chemical and biochemical reactions has a reaction chamber, an input aperture, and a lamp. A specimen receptacle with a reflective surface is positioned within the reaction chamber. The lamp has a light source, a temperature control system, and a lens. The light source is mounted onto the temperature control system and the lens is mounted onto the temperature control system. Accordingly, the temperature control system is able to moderate the temperature of the light source and the reaction chamber. The input aperture is an inlet that enables the lamp to be mounted onto the reaction chamber and retained in optical communication with the specimen receptacle. The positioning of the input aperture enables the light from the light source to be evenly distributed across an exterior surface of a specimen placed within the specimen receptacle.

### FIELD OF THE INVENTION

The present invention relates generally to a photo reactor. More specifically, the present invention relates to a photoreactor device that employs temperature control systems to moderate the temperatures reached during a photoreaction operation.

### BACKGROUND OF THE INVENTION

Over the past decade, the development and use of visible light-mediated photocatalysis have allowed the discovery of a wide variety of nontraditional bond-building in organic synthesis. See, e.g., the discussion at 81 J. Org. Chem. 6898-6926 (2016). Employing this low-cost energy source and ease of operation of visible light compared to a conventional UV photoreactor to promote reactivity and perform synthetic transformations has grown dramatically, with an increase in research labs developing visible light driven new synthetic methodologies.

In particular, the application of this technology to elucidate protein function within a cellular context is of great interest in chemical biology. See, e.g., the discussion at 76 Czech. Chem. Commun 859-917 (2011). This is because the use of visible light to induce photochemical reactions may allow for chemical transformations without perturbing the overall cellular environment. It is believed that due to the mild nature of visible irradiation compared to ultraviolet irradiation, photoredox chemistry will play an important role in understanding human biology.

Photoredox technology has become a very useful tool for making and breaking bonds not only in chemical biology but in material chemistry. However, the scope and performance of conventional single visible light driven reactions were has been found to be somewhat limited. To perform multiple array reactions or in cell protein labelling, a multi-well reactor setup which can be applied to visible light driven photoredox reaction is required.

### SUMMARY OF THE INVENTION

This array photoreactor of the present invention provides a simple and efficient platform for array screening synthesis in medicinal chemistry, and multiple wall plates for in-vitro cell labelling. The photoreactor has been engineered to provide uniform light penetration and equipped with temperature-controlled sink plates, to ensure that the reaction is performed at room temperature. The reactor also features an adjustable wavelength adsorption capability for most common photocatalysts or photosensitizers.

### BRIEF DESCRIPTION OF THE DRAWINGS

FIG. 1 is a front side perspective view of one embodiment of the photoreactor of the present invention, shown in an open position, that is, with the access door or hatch open.

FIG. 2 is an exploded perspective view of the photoreactor of FIG. 1.

FIG. 3 is a front view of the photoreactor of FIG. 1.

FIG. 4 is a rear view of the photoreactor of FIG. 1.

FIG. 5 is a top view of the photoreactor of FIG. 1 with the housing removed.

FIG. 6 is a flow diagram showing the electrical and electronic connections between the components used in the photoreactor of FIG. 1.

FIG. 7 is a front side perspective view of the photoreactor of FIG. 1 with the access hatch closed.

### DETAILED DESCRIPTION OF PREFERRED EMBODIMENTS

All illustrations of the drawings are for the purpose of describing selected versions of the present invention and are not intended to limit the scope of the present invention.

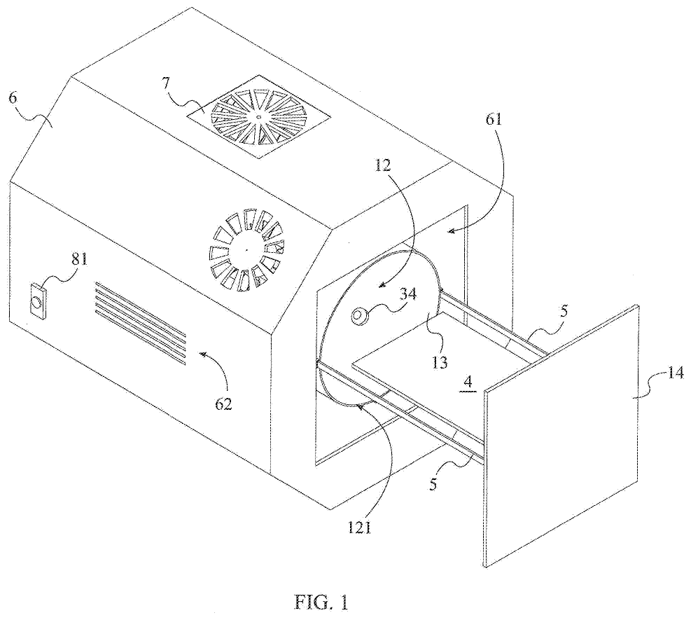

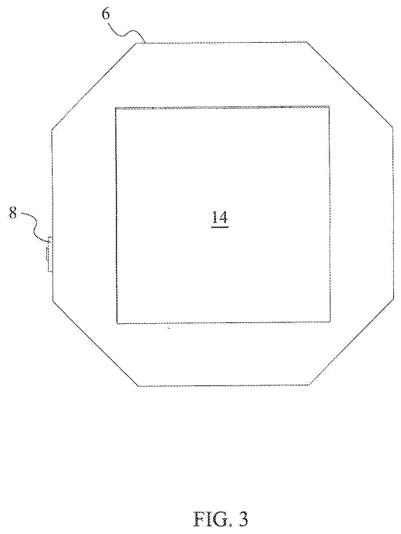

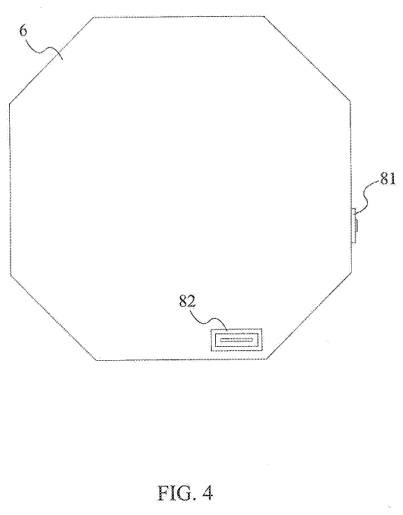

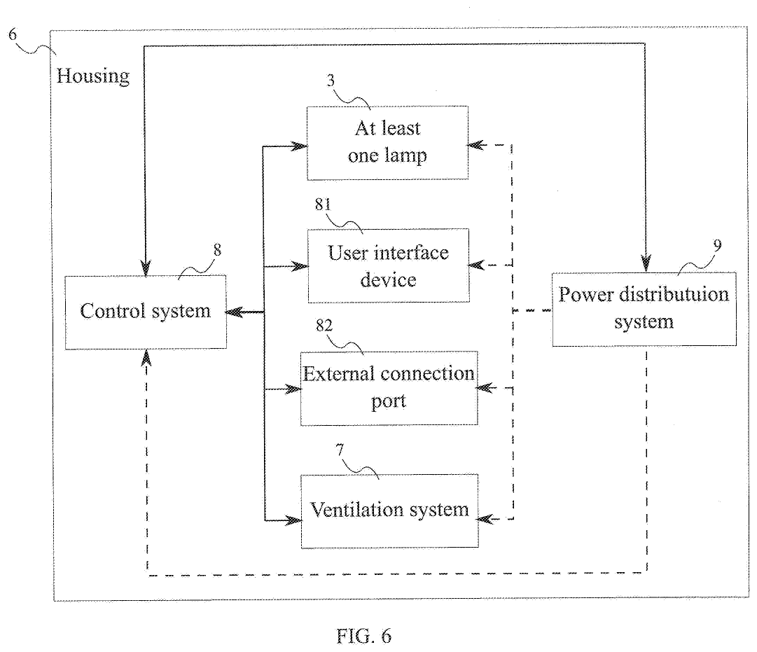

Referring to FIG. 1 through FIG. 7, the present invention, the photoreactor for cell assay and array chemical and biochemical reactions, is a device that uses light to facilitate the performance of chemical and biological reactions. To accomplish this, the present invention provides a reaction chamber that is designed to evenly distribute the light produced by one or more light sources across a chemical or biological specimen. Specifically, the present invention comprises a reaction chamber 1, at least one input aperture 2, and at least one lamp 3. The reaction chamber 1 is a rigid structure in which the chemical specimen is subjected to the light produced by the lamp 3. Additionally, the reaction chamber 1 comprises a chamber body 11, a specimen receptacle 12, and a reflective surface 13. The chamber body 11 forms the structural foundation of the reaction chamber 1 and defines the shape of the reaction chamber 1. Preferably, the chamber body 11 is cylindrical in shape. The specimen receptacle 12 traverses into the chamber body 11 so that the chemical specimen can be inserted into the reaction chamber 1 and exposed to light produced by the lamp 3. Additionally, the reflective surface 13 is superimposed onto the specimen receptacle 12. As a result, the reflective surface 13 facilitates simplifying and evenly distributing the light produced by the lamp 3. The input aperture 2 is a device that enables external light to enter the specimen receptacle 12. As such, the input aperture 2 is laterally integrated into the chamber body 11.

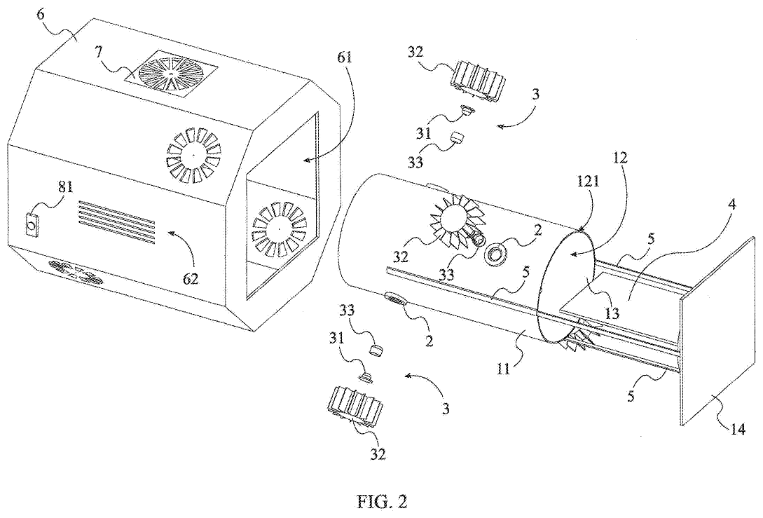

Referring to FIG. 1 and FIG. 2, the present invention is designed to produce highly tunable beams of light while remaining within a desired thermal profile. To accomplish this, the lamp 3 comprises a light source 31, a temperature control system 32, and a lens 33. Preferably, the light source 31 is an LED-based unit that produces a specific wavelength and intensity beam. However, the light source 31 can be adjustable system capable of producing light of varying wavelength, and intensity. The temperature control system 32 is preferably a phase-change heat sink. However, the temperature control system 32 can incorporate thermal modification devices that include, but are not limited to, fans, heatsinks, and chemical-reaction-based cooling devices. The light source 31 is mounted onto the temperature control system 32. Additionally, the lens 33 is mounted onto the temperature control system 32. Consequently, the lamp 3 forms a self-contained unit that produces at least one highly-tunable beam of light. The temperature control system 32 is mounted over the input aperture 2. Additionally, the light source 31 is optically coupled to the specimen receptacle 12 through the lens 33 and the input aperture 2. Accordingly, the input aperture 2 forms the inlet through which the highly-tunable light beam enters the specimen receptacle 12 after leaving the lamp 3. Additionally, the lens 33 is positioned to advantageously modify the highly-tunable beam of light before the highly-tunable beam of light enters the specimen receptacle 12 through the input aperture 2. Alternative embodiments of the present invention are designed with filters that further modify the characteristics of the highly-tunable beam of light during a photoreaction. The light source 31, the lens 33, the input aperture 2, and the specimen receptacle 12 are in thermal communication with the temperature control system 32. As a result, the temperature control system 32 is able to conduct thermal energy away from the lamp 3 and the specimen receptacle 12 during a reaction operation. This thermal conductivity enables the lamp 3 to produce high intensity beams of light without exiting a desired thermal profile.

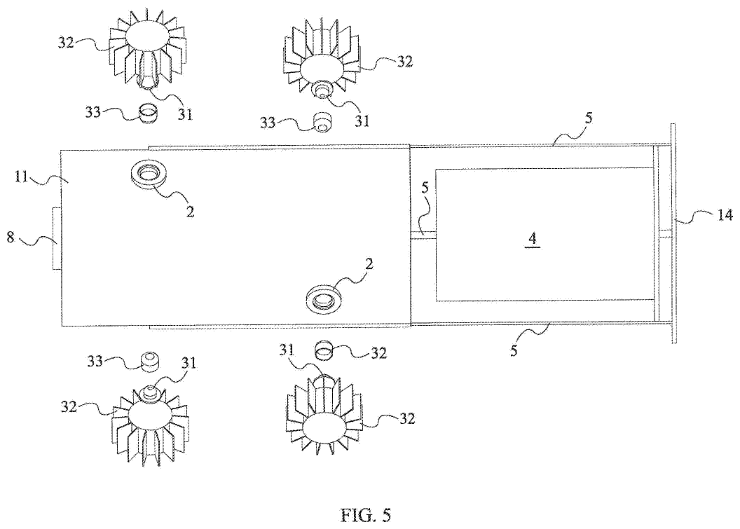

Referring to FIG. 1 and FIG. 5, the present invention is designed to enable a user to perform a wide range of photoreactions. To facilitate this, the lamp 3 is detachably mounted onto the reaction chamber 1. Specifically, the input aperture 2 is a coupling mechanism that is connected in between the lamp 3 and the chamber body 11. As a result, the lamp 3 can be removed or replaced as required. This enables the user to employ the most appropriate lamp 3 when performing a photoreaction. Preferably the input aperture 2 is a magnetic coupler that enables the lamp 3 to be quickly attached to or detached from the chamber body 11. The present invention is preferably designed with the at least one lamp 3 being a plurality of lamps 3 and the at least one input aperture 2 being a plurality of apertures 2. This arrangement enables each of the plurality of lamps 3 to be coupled to a corresponding aperture from the plurality of input apertures 2. Further, each of the plurality of input apertures 2 is advantageously positioned across the chamber body 11. Thus, enabling the highly-tunable beam generated by each of the plurality of lamps 3 to be evenly distributed within the specimen receptacle 12. Alternatively, the plurality of input apertures 2 is positioned such that he highly-tunable beam generated by each of the plurality of lamps 3 is focused onto one or more points within the specimen receptacle 12.

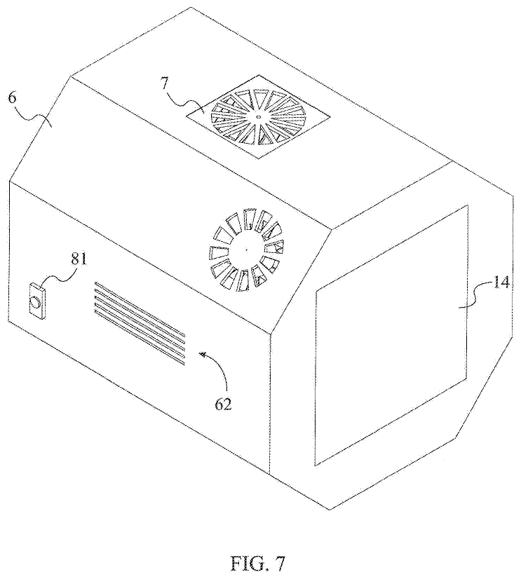

Referring to FIG. 1, FIG. 2, and FIG. 7, the present invention is designed to function as an enclosure in which sensitive reactions can occur. To achieve this functionality, the present invention comprises at least one rail assembly 5, a specimen retention system 4, and at least one access hatch 14. The rail assembly 5 is a movable support system that is designed to retain the chemical specimen in a desired position within the specimen receptacle 12. The specimen retention system 4 is a device that maintains the chemical specimen in a desired orientation within the specimen receptacle 12. Additionally, the specimen retention system 4 is designed to facilitate the light within the specimen chamber being evenly distributed across an exterior surface of the chemical specimen. The specimen retention system 4 can be any type of mounting device including, but not limited to, racks, clamps, and trays. The rail assembly 5 is laterally mounted onto the chamber body 11. Additionally, the rail assembly 5 is operatively coupled to the specimen retention assembly, wherein the rail assembly 5 slides the specimen retention system 4 into or out of the specimen receptacle 12. Accordingly, the rail assembly 5 acts as a support system that enables the user to mount the chemical specimen onto the specimen retention system 4 and while the specimen retention system 4 is positioned outside of the specimen receptacle 12.

Referring to FIG. 1, FIG. 2, and FIG. 7, the rail assembly 5 further enables to slide the loaded specimen retention system 4 into the specimen receptacle 12 in preparation of the chemical specimen undergoing the photoreaction. The user can then slide the specimen retention system 4 out of the specimen receptacle 12 along the rail assembly 5. The access hatch 14 is designed to selectively seal or unseal an opening of the specimen receptacle 12. Specifically, the rail assembly 5 is operatively coupled to the access hatch 14, wherein the rail assembly 5 slides the access hatch 14 closer to or away from an open end 121 of the specimen receptacle 12. Consequently, the access hatch 14 seals the open end 121 of the specimen receptacle 12 when the specimen retention system 4 is slid into the specimen receptacle 12. Once sealed, light is prevented from exiting the specimen receptacle 12. When the photoreaction is complete the user can unseal the access hatch 14 and slide the specimen retention system 4 out of the specimen receptacle 12.

Referring to FIG. 1, FIG. 2, and FIG. 5, the present invention is designed to function both as a self-contained unit that prevents the high temperatures caused by the photoreaction from damaging objects in the external environment. To achieve this functionality, the present invention further comprises a housing 6, at least one vent 62, and a ventilation system 7. The reaction chamber 1 is mounted within an interior compartment 61 of the housing 6. Preferably, the access hatch 14 seals both an opening of the interior compartment 61 and the open end 121 of the specimen receptacle 12 while the specimen retention system 4 is positioned within the specimen receptacle 12. As such, the reaction chamber 1 and the lamp 3 are relatively isolated from the external environment. The at least one vent 62 is laterally integrated into the housing 6 so that ambient air can be circulated between the interior compartment 61 and the external environment. Similarly, the ventilation system 7 is laterally integrated into the housing 6. Accordingly, the ventilation system 7 can force air to circulate within the interior compartment 61. Thus, regulating the temperature of the lamp 3 and the reaction chamber 1. Embodiments of the present invention are designed with a supplementary temperature control system 32 that further regulates the temperature within the interior compartment 61.

Referring to FIG. 1 through FIG. 6, the present invention is designed to function as both a stand-alone unit and while connected to an external computing device. To achieve this functionality, the present invention further comprises a control system 8, at least one user interface device (UID) 81, and a power distribution system 9. The control system 8 is an integrated processing unit that governs the function of the electrical components of the present invention. Additionally, the control system 8 is mounted within the housing 6 so that the control system 8 is protected from hazards in the external environment. Further, the control system 8 is electronically connected to the lamp 3, the UID 81, the external connection port 82, and the power distribution system 9. Accordingly, the control system 8 is able to monitor the state of and govern the operation of the aforementioned components. The UID 81 is laterally mounted onto the housing 6 to enable the user to interact with, and issue commands through, the UID 81 Preferably the UID 81 comprises a shutoff switch that enables the user to power the unit on or off. The external connection port 82 is an electrical interface that enables the present invention to be physically and electrically connected to an external system. Electrical power and data can be transmitted through the external connection port 82. Some embodiments of the present invention are designed with wireless communication systems that enable the present invention to be wirelessly connected to external computing devices. The power distribution system 9 is designed to provide for the electrical requirements of the present invention. As such, embodiments of the present invention are designed with power distribution systems 9 that comprise battery packs and external power inlets. The power distribution system 9 is mounted within the housing 6 and is electrically connected to the control system 8 and the lamp 3.

Although the invention has been explained in relation to its preferred embodiment, it is to be understood that many other possible modifications and variations can be made without departing from the spirit and scope of the invention as hereinafter claimed.

What is claimed is:

1-12. (canceled)

13. A visible light photoreactor for multiple specimens or samples for reaction, having a reactor space with uniform light distribution across and temperature control sufficient for reactions involving living cells, said photoreactor comprising:
at least one lamp comprising a lens, a light source associated with said lens, and a temperature control system comprising a heat sink associated with said light source, said light source having a specific and optionally adjustable light wavelength and intensity, and said lens positioned to distribute light uniformly across the reactor space;
a reaction chamber comprising a specimen receptacle, an interior reflective surface to facilitate uniform light distribution across the reactor space, and at least one aperture corresponding to each at least one lamp for mounting said lamp such that the light source and the temperature control system are external the reactor space;
a specimen retention system for maintaining specimens or samples in a desired orientation in the specimen receptacle;
a housing comprising a ventilation system, an operating control system for the at least one lamp, a power distribution system for the operating control system and having an external connection port, and a ventilation system that forces ambient air to circulate within the housing and that comprises at least one vent;
at least one hatch for access to and for closing the reaction chamber; and
at least one user interface device;
wherein said temperature control system and said ventilation system provide temperature control sufficient for reactions involving living cells.

14. The visible light photoreactor of claim 1 wherein the light source comprises an LED-based unit.

15. The visible light photoreactor of claim 1 wherein the reaction chamber is cylindrical in shape.

16. The visible light photoreactor of claim 1 wherein the at least one lamp is detachably mounted.

17. The visible light photoreactor of claim 16 wherein the at least one lamp is detachably mounted with a magnetic coupler.

18. The visible light photoreactor of claim 1 having a plurality of lamps across the reaction chamber.

19. The visible light photoreactor of claim 1 wherein the temperature control system further comprises a fan.

20. The visible light photoreactor of claim 1 wherein the heat sink is a phase-change heat sink.

21. The visible light photoreactor of claim 1 wherein the temperature control system further comprises a chemical-reaction-based cooling device.

22. The visible light photoreactor of claim 1 wherein the temperature control provided by the temperature control system and the ventilation system allows for reactions involving living cells at about room temperature.

23. The visible light photoreactor of claim 1 wherein the user interface device allows for wireless remote control of the photoreactor.

24. The visible light photoreactor of claim 1 wherein the specimen retention system can accommodate specimens or samples of different sizes and shapes at the same time.

25. The visible light photoreactor of claim 24 wherein the specimen or samples are in vials, or on trays or plates.

26. The visible light photoreactor of claim 1 further comprising a rail assembly laterally mounted onto the reaction chamber and operably coupled to the specimen retention system for sliding the specimen system into and out of the specimen receptacle and for providing a movable support system to retain the specimens or samples in a desired position within the specimen receptacle.

27. The visible light photoreactor of claim 22 wherein the temperature control provided by the temperature control system and the ventilation system allows for cell assay.

28. The visible light photoreactor of claim 22 wherein the temperature control provided by the temperature control system and the ventilation system allows for array chemical and biochemical reactions.
